(19) United States
(12) Patent Application Publication
(10) Pub. No.: US 2026/0207326 A1
(43) Pub. Date: Jul. 23, 2026

(54) TRANSFEMORAL SYSTEM FOR THE LOCALIZED TREATMENT OF AORTIC VALVE STENOSIS

(71) Applicant: AorticLab srl, Colleretto Giacosa (TO) (IT)

(72) Inventors: Enrico FERMI, Piacenza (IT); Enrico PASQUINO, Savigny (CH); Francesco BONETTI, Torino (IT); Franco OSTA, Pozzengo di Mombello Monferrato (AL) (IT)

(21) Appl. No.: 19/137,195

(22) PCT Filed: Oct. 26, 2023
(86) PCT No.: PCT/EP2023/079876

§ 371 Date: Jun. 9, 2025

(30) Foreign Application Priority Data
Dec. 12, 2022 (EP) 22212746.6

(51) Int. Cl. A61F 2/24; A61B 17/22

(52) U.S. Cl. CPC A61F 2/2418; A61B 17/2202; A61F 2/2433; A61F 2/2436; A61F 2250/0093

(57) ABSTRACT

comprising a working catheter (3) and at least one tool being configured to slidably move through the working catheter (3); characterized by the fact that said tool is an expandable stent (26) configured to allow an opening of an aortic native valve, the stent (26) furthermore comprising an ultrasonic transducer (25).

### FIELD OF INVENTION

The present invention relates to the treatment of aortic valve stenosis and more precisely to the treatment of calcified and/or fibrotic aortic valve leaflets, for instance by ultrasound.

### BACKGROUND

The early treatment of aortic stenosis, due to calcification of the valve leaflets or fibrosis, is essential to defer the timing of valve replacement operations (both thoracic or transcatheter), and to restore valve conditions in inoperable subjects.

Delaying the implantation of an aortic valve prosthesis, for a few years, allows to improve the patient's quality of life as well as cost savings, thanks to the prevention of secondary pathologies or complications related to the artificial valve implant. It also reduces the risk of having to resort to a subsequent valve-in-valve TAVR implant.

Some mechanical solutions have been proposed such as valvuloplasty, using a balloon catheter, or based on scoring of the calcific leaflets to create fractures in the calcific deposits.

Valvuloplasty presents potential risks such as accidental rupture of the cusps, while both techniques have the major limitation of a reduced duration of efficacy of the treatment, in reducing valve stenosis, due to a rapid recalcification, already starting from the first months following treatment.

Other proposed solutions are based on the use of ultrasound obtained with impulse signals, capable of fragmenting the calcific deposits with small shockwaves emitted in the immediate vicinity of the aortic valve cusps.

See for example patent application EP 3 685 772 A1 “Device for the treatment of the calcification of tissues”.

All proposed systems use a catheter that is introduced from the femoral artery, until it reaches the valve in the aortic area.

The simultaneous presence, in the catheter, of electrical connection cables, insulation systems, guide wires, temporary artificial valves and any other structures, requires a necessary reduction in the dimensions of the active ablation elements to the detriment of the effectiveness of the treatment. The width of the surface of the ultrasound sources affects both the acoustic intensity of the ultrasound field and the size of the treated valve area.

### GENERAL DESCRIPTION OF THE INVENTION

The present invention provides a transfemoral system for the localized treatment of aortic valve stenosis comprising a working catheter and at least one tool being configured to slidably move through the working catheter. The system according to the invention advantageously comprises several tools that can be alternatively used within the same working catheter. Consequently, the dimensions of each tool may be maximized.

The invention more precisely concerns a transfemoral system, as defined in the claims, for the localized treatment of aortic valve stenosis

According to one embodiment of the invention, the system is characterized by the fact that at least one tool is an expandable stent configured to allow an opening of an aortic native valve. The stent furthermore contains at least one ultrasonic transducer.

In a general manner, the system according to the present invention includes:
a first working catheter, for navigation, to reach the aortic site from the peripheral femoral artery. Preferably the working catheter includes an inflatable and retractable tip, and advantageously a guide wire, inserted through the tip;
preferably several different tools tailored to the treatment of the specific problems that cause valve stenosis aspect of aortic valve stenosis, wherein each tool can be inserted into or retracted from the working catheter.

The following three tools can be used in the system of the present invention, only one of them, two or all three:
A tool comprising ablation units based on rectangular piezoceramic transducers, for the fragmentation of intra-tissue calcium deposits or the treatment of valve fibrosis.
As part of this tool, a particular support structure for these units allows them to be positioned precisely on the area of the leaflets to be treated, being able to rotate, flex and extend, keeping the distance between the transducers fixed, to create an emission surface equal to the sum of the surfaces of each single transducer. It also allows the transducers to follow the irregular profile of the valve leaflet that is thickened because of calcified deposits or due to fibrosis.
Another tool based on mechanical percussion and vibration units for the specific treatment of fusion of the commissures.
A third tool including an expandable stent which, once positioned in the patient's native valve, may gently force its opening. Preferably one or several cylindrical ultrasonic transducers are used. They support the opening action of the stenotic valve, breaking down calcium, if present, or remodelling the tissue if in presence of fibrosis.

Other tools may also be used in the system of the present invention. The working catheter is preferably made of biocompatible material, for example polymeric, such as, but not limited to, polyamide, Grilamid or Vestamid, or equivalent materials, and has an external diameter of less than 18 fr, or in any case not greater than 21 fr.

### DETAILED DESCRIPTION OF THE INVENTION

The invention will be better understood in the present chapter, with some non-limiting examples illustrated by the following figures:

FIG. 1—Movement of the transcatheter system in the aortic arch

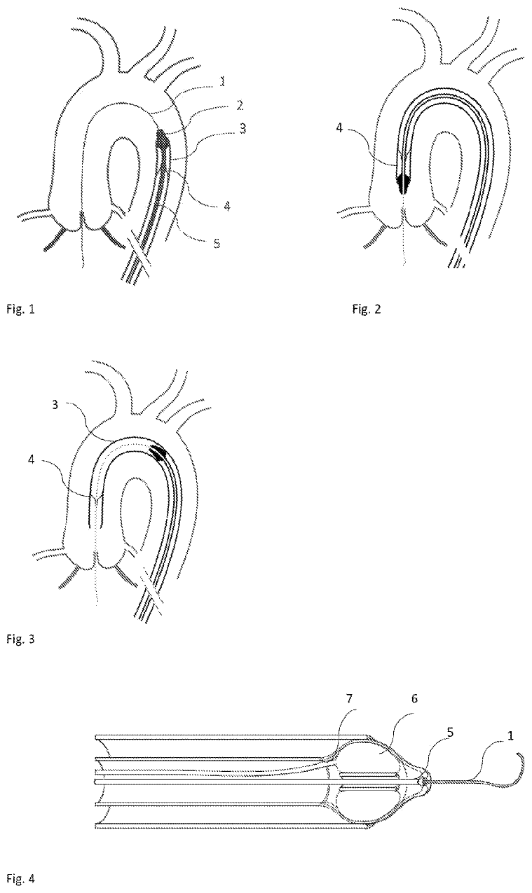

FIG. 2—Movement of the transcatheter system in the aortic arch, reaching the native valve site

FIG. 3—Deflated tip and retrieve through the navigation catheter which remains in place

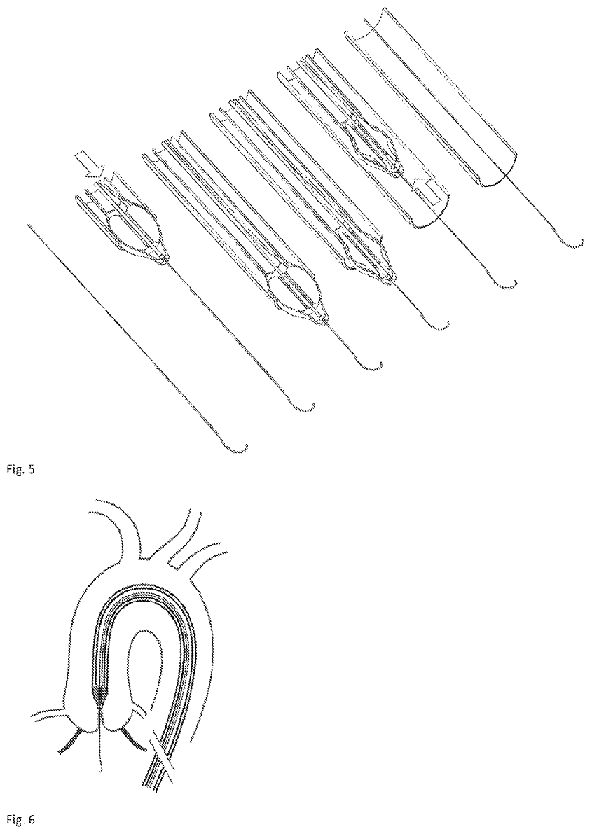

FIG. 4—Tip with inflatable chamber and hypotube

FIG. 5—Sequence of device placement and retrieve leaving the navigation catheter in place

FIG. 6—Placement of of the valvular treatment tools within the navigation catheter

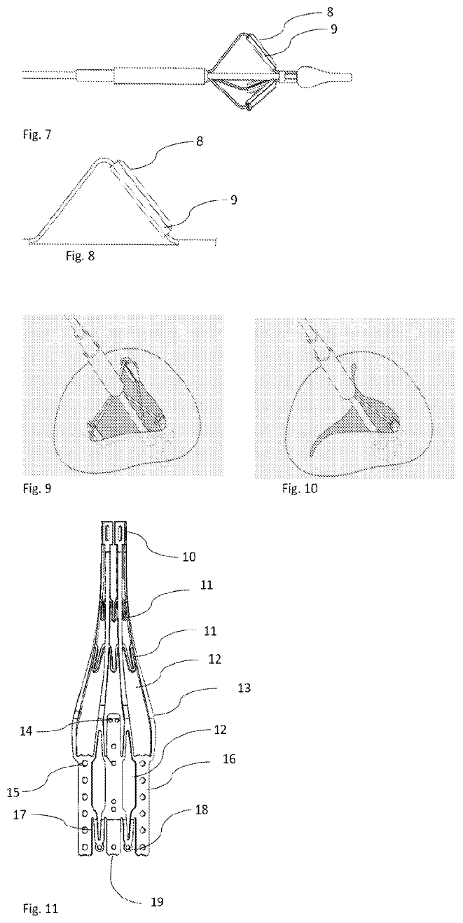

FIG. 7—Variant of the tool with 3 strikers for the treatment of valve with fused or partially fused commissures

FIG. 8—Variant of the tool with a single striker for the treatment of valve with fused or partially fused commissures

FIG. 9—Placement of the 3-striker tool in the native aortic valve

FIG. 10—Placement of the single striker tool in the native aortic valve

FIG. 11—Support structure for the ablation units

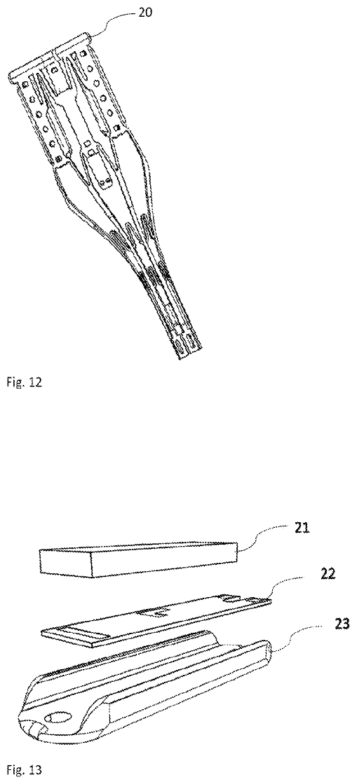

FIG. 12—Support structure for the ablation units with protective skirt

FIG. 13—Exploded view drawing of an ablation unit that comprises piezoceramic or polymer-based piezoelectric translator, fpc for electrical connection and isolation, metal support.

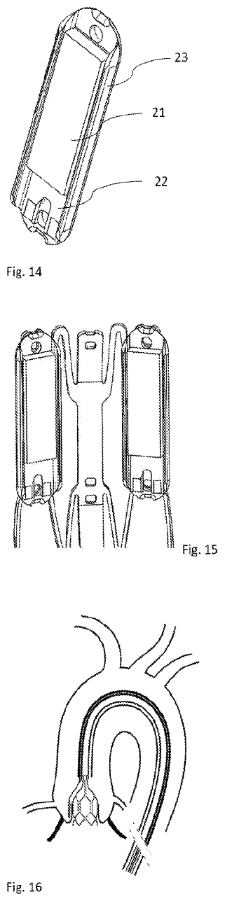

FIG. 14—Piezoceramic or polymer-based piezoelectric rectangular transducer mounted on metal support with fpc for electrical connection

FIG. 15—Ablation units mounted on a support structure

FIG. 16—Positioning of the ablation tool and temporary artificial valve

FIG. 17—Ablation unit and stent support structure

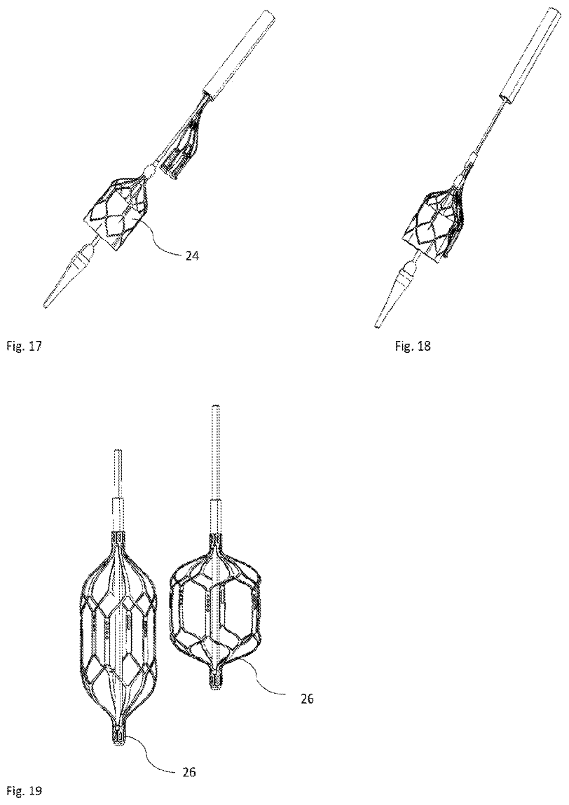

FIG. 18—Ablation unit support structure overlaid with the stent

FIG. 19—Expandable stent (without integrated artificial valve and without ultrasound emitters)

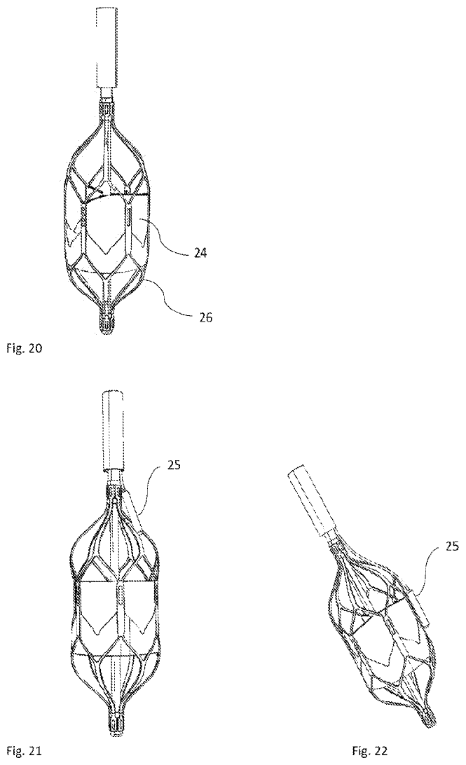

FIG. 20—Expandable stent with artificial valve

FIG. 21—Expandable stent with artificial valve and initial deployment of a “cylindrical ultrasound emitter (tube)”

FIG. 22—Expandable stent with artificial valve and final positioning of a “cylindrical ultrasound emitter (tube)”

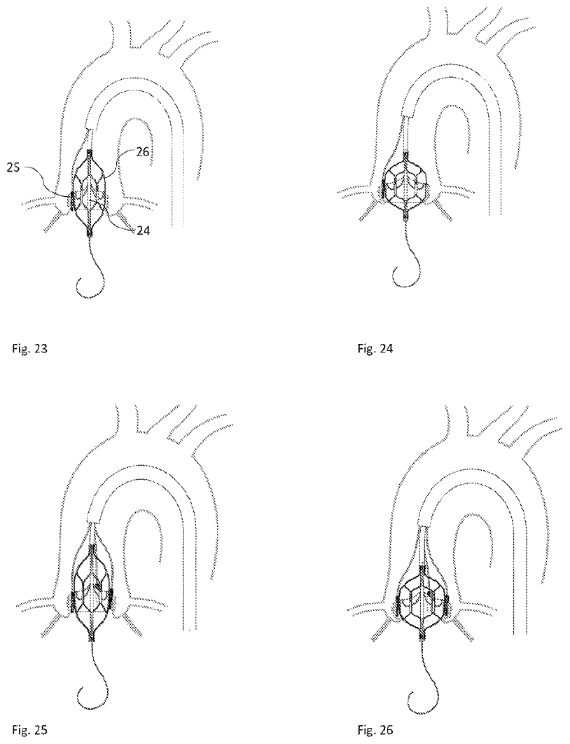

FIG. 23—Expandable stent with artificial valve with one transducer outside the stent, open in the native valve: the structure is not expanded

FIG. 24—Expandable stent with artificial valve with one transducer outside the stent, open in the native valve: the structure is expanded

FIG. 25—Expandable stent with artificial valve with three transducers outside the stent, open in the native valve: the structure is not expanded

FIG. 26—Expandable stent with artificial valve with three transducers outside the stent, open in the native valve: the structure is expanded

FIG. 27—Expandable stent with artificial valve with one transducer inside the stent, open in the native valve: the structure is not expanded

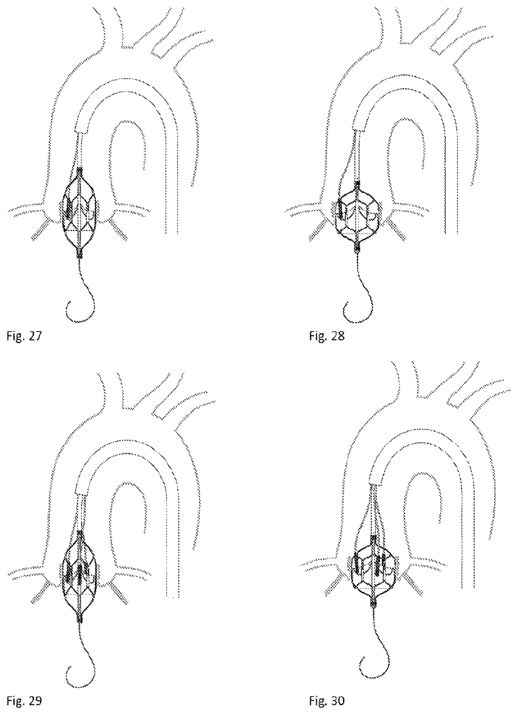

FIG. 28—Expandable stent with artificial valve with one transducer inside the stent, open in the native valve: the structure is expanded

FIG. 29—Expandable stent with artificial valve with three transducers inside the stent, open in the native valve: the structure is not expanded

FIG. 30—Expandable stent with artificial valve with three transducers inside the stent, open in the native valve; the structure is expanded.

### NUMERICAL REFERENCES USED IN THE FIGURES

1 Guidewire
2 Tip with inflatable/deflatable chamber
3 Working catheter
4 One-way blood valve
5 Hypotube
6 Inflatable chamber
7 Insufflation tube
8 Metal wedge
9 Linear Resonant Actuator (LRA)/vibration motor
10 Catheter coupling end
11 Strut connectors
12 Support cells
13 Tethering structures
14 Holes to tie the strands
15 Holes to fix transducer housing
16 Support legs
17 Struts in skirt region
18 Eyelet on strut
19 Central support
20 Protective flange
21 Ultrasound transducer
22 FPC flexible printed circuit
23 Transducer housing
24 Artificial valve
25 Piezoelectric tube (ultrasound emitter)
26 Expandable stent

As shown in FIGS. 1, 2 and 3, the procedure for positioning the working catheter 3 involves the use of a guidewire 1 and a the insertion of the device equipped with a tip 2 and hypotube 5 in which the guidewire 1 may slide.

According to one preferred embodiment of the present invention, the working catheter 3 is combined with a removable tip 2 made of inflatable, biocompatible plastic material, such as, but not limited to, silicone, polyurethane, polytetrafluoroethylene.

The tip 2, which can be expanded by means of a liquid inserted via a syringe through an insufflation tube 7, is inserted into the working catheter 3 and inflated, in the distal section of the catheter, until it is fully expanded, as shown in FIG. 4.

In this way, the tip 2 adheres perfectly to the walls of the catheter 3, preventing blood from flowing into the catheter 3.

The tip 2 and the working catheter 3 can be coated with a hydrophilic solution to facilitate tracking through artery, reducing friction with blood vessels.

Once the aortic site is reached, as shown in FIG. 2, the tip chamber is deflated with a consequent reduction in the size of the tip 2, which can be retracted and extracted from the working catheter 3, as shown in FIG. 3. The entire sequence is shown in FIG. 5.

Inside the working catheter 3, a hemostatic valve 4, for example a duckbill type or equivalent, prevents blood reflux when the tip 2 is retracted, waiting for the insertion of a tool within the working catheter 3. See FIG. 3 and FIG. 6.

The three aortic valve stenosis treatment tools shown in those examples are of several different types of construction, the first two being dedicated to the treatment of calcified cusps and commissures and the third to the opening of the fused commissures because of calcification.

The embodiment of the first tool uses at least two piezoceramic or polymer-based piezoelectric transducers 21 for the emission of an ultrasonic field capable of fragmenting the calcium deposits in the valve cusps or modifying the structure of the cusps thanks to the microcavities induced by cavitation. This structural modification changes the pliability of the leaflets, which become more flexible.

The transducers 21 are housed in metal supports 23 capable of safeguarding them from breaking and providing the backing function for focusing the emitted ultrasound field (FIGS. 13 and 14).

The set composed of a piezoceramic or polymer-based piezoelectric transducer 21 and metal support 23 constitutes an ablation unit.

The electrical connection with the transducer terminals is obtained by means of a flexible FPC circuit 22 placed between the transducer housing 23 and the transducer 21. This FPC 22 is made of polyamide with two exposed pads for electrical connection. The FPC 22 therefore also simultaneously provides electrical insulation, apart from the soldering pads, with the transducer 21.

It is necessary to consider the acoustic impedance coupling effects of the transmitting transducer face and those due to the presence of the backing medium in contact with the parallel face behind it.

Inside the transducer 21, the stress waves induced by the application of an electrical signal are generated at the center of the transducer 21 and propagate towards the faces that delimit it at the two ends.

The wave generated towards the backing interface is totally reflected towards the center of the transducer 21, adding to the main wave. Ideally, the amplitude of the wave emitted by the transducer surface is exactly double that which would occur in the transmission medium without reflective backing.

If the emitting surface of the transducer 21 is coupled to the transmission medium, not directly, but through an appropriate layer, called matching layer, with thickness λ\4, the conditions of maximum emission are achieved (the acoustic impedance should be equal to the square root of the product between the characteristic acoustic impedance of the transducer 21 and the acoustic impedance of the medium, such as blood or water Z_{M}=\sqrt{ZₚZ_{T}}) .

For applications where the transducer 21 must operate in an impulsive regime, it is necessary that the ultrasound pulse generated is made as short as possible, to avoid unwanted overlaps between direct impulse (in transmission) and reflected impulse (in reception).

Shorter pulses are obtained by applying a material with strong internal damping to the rear face. The acoustic impedance of the backing should be very similar to that of the transducer and to avoid loss of efficiency usually between 3 and 7 MRayl (e. g. resins loaded with metal powders and metal oxides, heraldite or epoxy cements).

To achieve good energy transmission from a normal piezoelectric transducer used for ultrasound generation, characterized by a high characteristic acoustic impedance, to biological tissues, which have a relatively low impedance, a layer of coupling medium is generally used (matching layer) of appropriate material, so-called “quarter wavelength (λ/4)”.

For ablation units this layer corresponds to the thickness of the insulation resin layer.

The backing is instead obtained with a material of multiple thickness of λ/4. To obtain this effect, at the operating frequencies, the base of the metal supports of the piezoceramic transducers must be a few tenths of a millimeter.

A peculiar metal structure (FIG. 11) provides support for the ablation units. This structure also serves as a mechanical constraint, as shown in FIG. 11, so that the inter-distance between the transducers is predefined and kept fixed, to create an emission surface equal to the sum of the surfaces of each individual transducer. It also allows the transducers to follow the irregular profile of the valve leaflet that is thickened because of calcified deposits or due to fibrosis.

This structure (FIG. 11) is made of metal alloy with shape memory effect (SME), such as eg. Nitinol NiTi and includes:
a proximal tethering structure 13 with 4 arms with holes 10 for fixing to a hypotube 5 inside the vascular catheter;
strut connectors 11 for connection between the arms to ensure adequate strength for the structure;
two lateral support legs 16, for fixing the ablation units (FIG. 14), comprising of transducer 21+FPC 22+metal support 23;
a central support 19 for housing a temperature sensor (for example a PT100 RTD, resistive, or a thermocouple). Some holes 14 on this central support 19 allow to fix elements such as tie wires made of steel or Kevlar or high grade polyester, ... ) in order to flex or extend the entire structure during positioning on the native heart valve to be treated.

The terminal part of this structure (skirt region) includes two struts 17 with eyelets 18 for fixing a protective flange 20 made of polymer or other equivalent material. This protection saveguards the leaflets during the placement procedure of the ablation units.

This structure can rotate 360° allowing the control of the position of the ablation units with respect to the aortic valve to be treated. The electrical connection is made via a flexible polyamide FPC 22 printed circuit with gold plated pads, soldered to the silver armature of the transducer with solder paste or conductive resin. The FPC also simultaneously provides electrical insulation with respect to the transducer support (made of metal and therefore conductive).

The multi-layer structure in FIG. 13, includes the piezoceramic or polymer-based piezoelectric transducer 21, usually rectangular in shape, the metal support 23 and the electrical connection FPC 22 in the contact windows and at the same time for electrical insulation.

The electrical insulation of the entire ablation unit can be achieved through biocompatible coatings, such as Parylene-C or, preferably, polyurethane which allows good deformability of the piezoceramic or polymer-based piezoelectric transducers, excellent electrical insulation and mechanical resistance to avoid flaking as it slides into the catheter.

Through the working catheter 3 the ablation units reach the aortic valve site, where they emit impulsive ultrasonic fields at two frequencies, 3-4 MHz and 100-200 KHz.

These pulses have the form of low-power shock waves, with a positive peak followed by a negative one, with an amplitude about ¼ of the positive one, lasting about 20-60 ns.

Once the aortic site is reached, the ablation units come out of the catheter 3 and open, taking the shape of a trident with the ablation units on the side arms as in FIG. 17, remaining side by side.

This configuration allows ultrasound to be delivered over a large portion of the calcific aortic valve leaflets, increasing the effectiveness of the treatment. The acoustic power, in fact, depends on the area of the ultrasound emission unit. Two piezoceramic transducers are kept side by side so increasing the emission surface.

The solution includes a temporary artificial valve 24 made of polymeric material or fabric dipped with polyester, for example, as already described in international patent application WO 2020/151995 A1. The treatment tool based on these transducers is positioned by placing the ablation units on the patient's native valve leaflets, held open by the artificial valve (FIG. 18).

The solution also provides a specialized tool to treat the fused valve commissures (FIGS. 7 and 8). The ablation unit of this third type includes a single-axis haptic vibration motor 9, called LRA (Linear Resonant Actuator), to which a high hardness triangular wedge 8 is attached. Possible materials that can be used to make the triangular wedge are, for example: steel, titanium, ceramic.

This wedge-shaped striker has the function of a “chisel” able to open the commissures, in advance or even simultaneously, to the treatment with the ultrasound module.

The LRA motor is very compact in size, slightly smaller than the size of the delivery catheter.

This unit, comprising both the LRA motor and the triangular wedge, is positioned on an arm of a steel or nitinol structure which, when open, has the shape of a V, as shown in FIG. 8, while it is rectilinear when closed in the delivery catheter.

The catheter that includes this unit, reaches the aortic valve site, passing through the previously placed working catheter.

By retracting the covering sheath, the structure opens assuming an “inverted” V shape. The ablation unit, placed on the lower arm of this structure is positioned directly in the commissure to be treated (FIG. 10). The mechanical vibration generated by the motor and the consequent percussion imposed by the triangular wedge, promote the opening of the commissure partially closed or fused by calcification.

In a variant of the solution, three strikers are used, each positioned on an arm of the structure, as shown in FIG. 7. This variant treats the three valve commissures simultaneously (FIG. 9).

A third treatment tool for aortic valve stenosis includes the use of a stent 26 capable of expanding, (FIG. 19), once positioned in the patient's native valve to gently “force” the opening of the latter. This soft opening of the valve is obtained thanks to the stent 26 distally decreasing cross-section. In one embodiment, in addition to the mechanical action, the expandable stent tool includes at least one cylindrical piezoceramic transducer 25, or tube-shaped, for the emission of the ultrasound field (FIG. 20). Compared to a valvuloplasty that has the risk of valve rupture, the expansion achieved by this instrument, is slow and controlled and supported by the combined action of ultrasounds that break down calcium, if present, or remodel the tissue if in the presence of fibrosis.

The choice of using a cylindrical “tube-shaped” transducer, for this configuration, allows to emit the ultrasound field radially, allowing its simultaneous action on the leaflets and on the commissures, even if of lower intensity than the rectangular transducers used for the ablation tools previously described. However, the intensity is sufficient if used as an integrated action to the mechanical expansion action of the stent 26: the combination of gradual expansion and reshaping action of ultrasound increases the diameter of the native valve, reducing the obstruction of blood flow.

Variants Of this structure include: the use of a transducer 25 external to the stent 26 (FIG. 23-24), the use of 3 transducers external to the stent 26 (FIG. 25-26), the use of a transducer internal to the stent and external to the artificial valve (FIG. 27-28), the use of three transducers internal to the expandable stent (FIG. 29-30) and external to the artificial valve.

1. A transfemoral system for the localized treatment of aortic valve stenosis comprising:
a working catheter and at least one tool configured to slidably move through the working catheter;
wherein said tool is an expandable stent with a distally decreasing cross-section configured to allow an opening of an aortic native valve, the stent furthermore comprising an ultrasonic transducer.

2. System according to claim 1 wherein the ultrasonic transducer has a cylindrical shape.

3. System according to claim 1 wherein the stent comprises several ultrasonic transducers.

4. System according to claim 1 wherein the ultrasonic transducer is external to the stent.

5. System according to claim 1 wherein the ultrasonic transducer is internal to the stent.

6. System according to claim 1 wherein the stent contains an artificial valve.

7. System according claim 1 wherein the working catheter includes an inflatable and retractable tip.

8. System according to claim 1, further comprising another tool configured to slidably move through the working catheter; wherein said tool comprises a support structure and rectangular ultrasonic transducers fixed to the support structure; said support structure being configured to directly position said rectangular ultrasonic transducers on valve leaflets to be treated.

9. System according to claim 1 comprising another tool configured to slidably move through the working catheter; wherein said tool comprises mechanical percussion and vibrations units that are configured for the treatment of valve commissures.

10. System according to claim 9 wherein each mechanical percussion and vibration unit comprises a Linear Resonant Actuator equipped with a metal wedge capable of opening valve commissures.
